(19) United States
(12) Patent Application Publication
(10) Pub. No.: US 2022/0276200 A1
(43) Pub. Date: Sep. 1, 2022

(54) CHEMICAL SENSOR MODULE AND METHOD FOR DETECTING HYDROPHOBIC TARGET MOLECULES

(71) Applicant: KABUSHIKI KAISHA TOSHIBA, Tokyo (JP)

(72) Inventors: Yoshiaki SUGIZAKI, Fujisawa Kanagawa (JP); Hiroshi HAMASAKI, Hiratsuka Kanagawa (JP)

(73) Assignee: KABUSHIKI KAISHA TOSHIBA, Tokyo (JP)

(21) Appl. No.: 17/445,307

(22) Filed: Aug. 17, 2021

(30) Foreign Application Priority Data
Mar. 1, 2021 (JP) 2021-031476

(51) Int. Cl. G01N 27/414; G01N 33/00; G01N 1/38; G01N 33/58

(52) U.S. Cl. CPC G01N 27/4146; G01N 33/0031; G01N 33/0047; G01N 1/38; G01N 33/58; G01N 2001/386

(57) ABSTRACT

A chemical sensor module includes a target molecule uptake unit that exposes a sample atmosphere containing hydrophobic target molecules to a hydrophilic organic solvent; a mixing unit that mixes the organic solvent containing the target molecules with an aqueous solution to prepare a sample solution; and a sensor element having a surface exposed to the sample solution.

### CROSS-REFERENCE TO RELATED APPLICATION

This application is based upon and claims the benefit of priority from Japanese Patent Application No. 2021-031476, filed on Mar. 1, 2021; the entire contents of which are incorporated herein by reference.

### FIELD

Embodiments described herein relate generally to a chemical sensor module and a method for detecting hydrophobic target molecules.

### BACKGROUND

In a chemical sensor that detects target molecules incorporated into an aqueous solution from the gas phase in the aqueous solution, the target molecules existing in the gas phase are often hydrophobic and there is a concern that the detection sensitivity may decrease.

### BRIEF DESCRIPTION OF THE DRAWINGS

FIG. 1 is a schematic configuration diagram of a chemical sensor module of an embodiment;

FIG. 2 is a schematic configuration diagram of a chemical sensor module of the embodiment;

FIG. 3 is a schematic view showing an example of a target molecule uptake unit of the chemical sensor module of the embodiment;

FIG. 4 is a schematic view of another example of a target molecule uptake unit of the chemical sensor module of the embodiment;

FIG. 5 is a schematic perspective view of a flow path chip of the target molecule uptake unit shown in FIG. 4;

FIG. 6 is a schematic perspective view of a sensor element of the chemical sensor module of the embodiment;

FIGS. 7 to 10B are schematic views showing a detection mechanism of a target molecule in the chemical sensor module of the embodiment;

FIGS. 11A to 12B are schematic views showing a detection mechanism of the target molecule in a comparative example;

FIGS. 13A to 15B are schematic views showing another example of a detection mechanism of the target molecule in the chemical sensor module of the embodiment;

FIG. 16A is a graph showing a detection result of limonene by an experiment of the embodiment;

FIG. 16B is a graph showing a detection result of limonene by a control experiment; and

FIGS. 17A and 17B are schematic views of a sensor element mounting portion of the chemical sensor module of the embodiment.

### DETAILED DESCRIPTION

According to one embodiment, a chemical sensor module includes a target molecule uptake unit that exposes a sample atmosphere containing hydrophobic target molecules to a hydrophilic organic solvent; a mixing unit that mixes the organic solvent containing the target molecules with an aqueous solution to prepare a sample solution; and a sensor element having a surface exposed to the sample solution.

Hereinafter, the embodiments will be described with reference to the drawings as appropriate. For the convenience of explanation, the scale of each drawing is not always accurate and may be indicated by a relative positional relationship or the like. Further, the same or similar elements are designated by the same reference numerals.

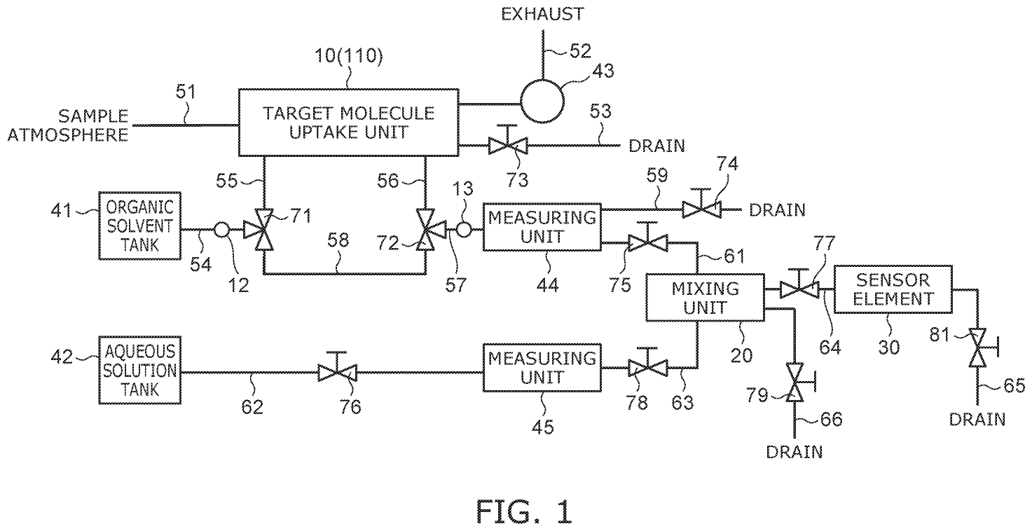

FIG. 1 is a schematic configuration diagram of a chemical sensor module of an embodiment.

The chemical sensor module of the embodiment includes at least a target molecule uptake unit 10, a mixing unit 20, and a sensor element 30.

The target molecule uptake unit 10 is connected to a pipe and a pipe 52. An intake and exhaust device 43 is connected to the pipe 52. The intake and exhaust device 43 is, for example, a pump or a fan. By driving the intake and exhaust device 43, the sample atmosphere is taken into the target molecule uptake unit 10 via the pipe 51. The detection target in the chemical sensor module of the embodiment is hydrophobic target molecules contained in the sample atmosphere.

The target molecule uptake unit 10 is connected to a supply source of an organic solvent. For example, the target molecule uptake unit 10 is connected to an organic solvent tank 41 in which an organic solvent is stored via a pipe 54, a pipe 55, and a valve 71. The organic solvent is a hydrophilic organic solvent and is selected from the group consisting of, for example, lower alcohols such as ethanol and methanol, DMSO (dimethyl sulfoxide), DMF (N,N-dimethylformamide), acetone, and acetonitrile.

The organic solvent is supplied from the organic solvent tank 41 to the target molecule uptake unit 10. The target molecule uptake unit 10 exposes the sample atmosphere, which may contain hydrophobic target molecules, to the hydrophilic organic solvent.

The target molecule uptake unit 10 is connected to a pipe 53 for draining the liquid and a valve 73 is connected to the pipe 53. Further, the target molecule uptake unit 10 is connected to a measuring unit 44 via a pipe 56, a valve 72, and a pipe 57.

The organic solvent tank 41 is connected to the measuring unit 44 via the pipe 54, the valve 71, a pipe 58, the valve 72, and the pipe 57.

The measuring unit 44 is connected to a pipe 59 for draining the liquid and a valve 74 is connected to the pipe 59. Further, the measuring unit 44 is connected to the mixing unit 20 via a pipe 61 and a valve 75 is connected to the pipe 61.

Further, a supply source of an aqueous solution is connected to the mixing unit 20. For example, the mixing unit 20 is connected to an aqueous solution tank 42 in which an aqueous solution is stored via a measuring unit 45. A valve 76 is connected to a pipe 62 that connects the aqueous solution tank 42 and the measuring unit 45. A valve 78 is connected to a pipe 63 that connects the mixing unit 20 and the measuring unit 45. The aqueous solution is, for example, a phosphate buffer, a HEPES buffer, a Tris-hydrochloride buffer, or the like.

The mixing unit 20 is supplied with an organic solvent containing target molecules from the target molecule uptake unit 10 and also supplied with an aqueous solution from the aqueous solution tank 42. Then, the mixing unit 20 mixes the organic solvent containing target molecules with the aqueous solution to prepare a sample solution.

The mixing unit 20 is connected to the sensor element 30 via a pipe 64. A valve 77 is connected to the pipe 64. Further, the mixing unit 20 is connected to a pipe 66 for draining the liquid and a valve 79 is connected to the pipe 66.

The sensor element 30 is connected to a pipe 65 for draining the liquid and a valve 81 is connected to the pipe 65.

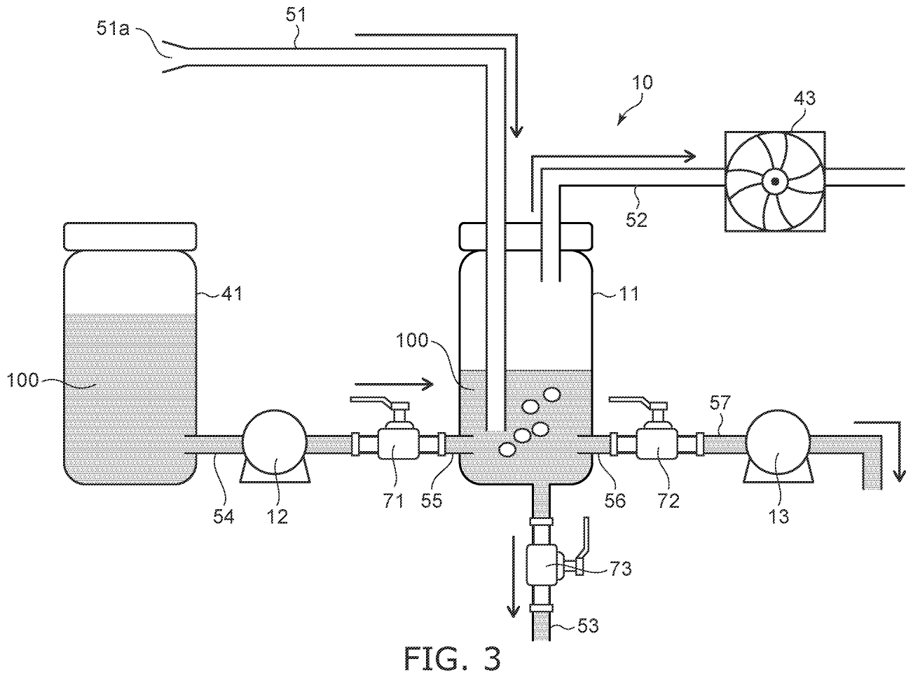

FIG. 3 is a schematic view showing an example of the target molecule uptake unit 10.

The target molecule uptake unit 10 includes a tank 11 for bubbling the sample atmosphere with an organic solvent. The tank 11 is connected to the organic solvent tank 41 via the pipe 54, the valve 71, and the pipe 55. A pump 12 is connected to the pipe 54. By opening the valve 71 toward the pipe 55 and driving the pump 12, an organic solvent 100 stored in the organic solvent tank 41 is supplied into the tank 11.

At one end of the pipe 51, an atmosphere collection port 51a located outside the tank 11 is formed. The other end of the pipe 51 is located in the organic solvent 100 in the tank 11. Further, one end of the pipe 52 is located in the gas phase part above the organic solvent 100 in the tank 11, and the other end of the pipe 52 is an exhaust port, and the intake and exhaust device 43 is connected in the middle of the tank 11 and the exhaust port. By driving the intake and exhaust device 43, the sample atmosphere taken into the pipe 51 from the atmosphere collection port 51a is bubbled with the organic solvent in the tank 11, and the target molecules in the sample atmosphere are dissolved in the organic solvent.

The tank 11 is connected to the measuring unit 44 described above via the pipe 56, the valve 72, and the pipe 57. By opening the valve 72 toward the pipe 56 and driving a pump 13, the organic solvent containing target molecules in the tank 11 is supplied to the measuring unit 44.

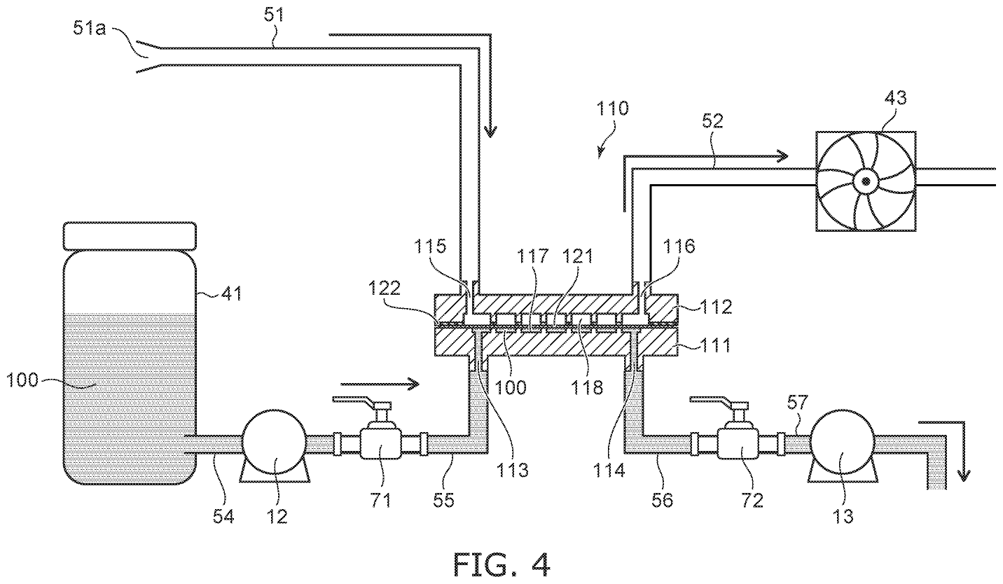

FIG. 4 is a schematic view of a target molecule uptake unit 110 of another example.

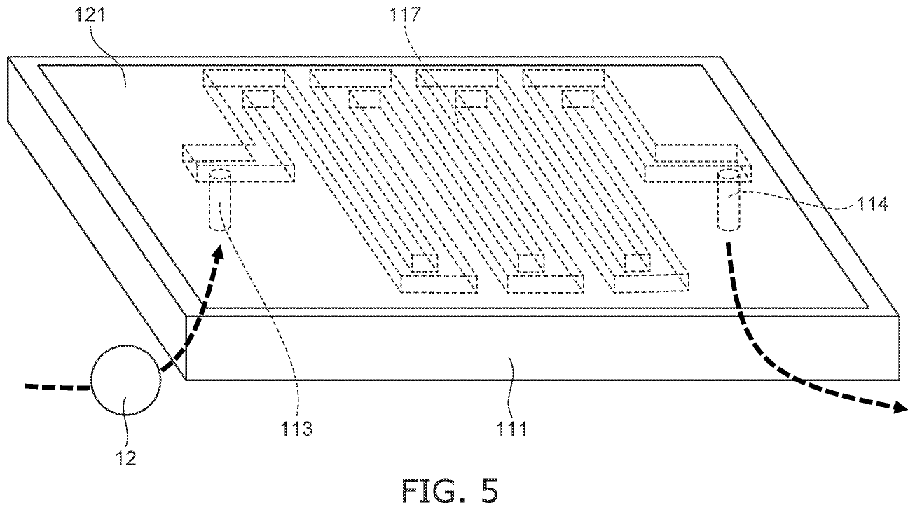

FIG. 5 is a schematic perspective view of a flow path chip 111 of the target molecule uptake unit 110 shown in FIG. 4.

The target molecule uptake unit 110 includes the flow path chip 111, a lid 112 superposed on the flow path chip 111, and a porous membrane 121 disposed between the flow path chip 111 and the lid 112.

As shown in FIG. 5, a groove 117 is formed on the upper surface of the flow path chip 111. Further, the flow path chip 111 is formed with a liquid inflow path 113 connected to one end of the groove 117 and a liquid outflow path 114 connected to the other end of the groove 117. As shown in FIG. 4, the liquid inflow path 113 is connected to the pipe 55 to which the organic solvent is supplied, and the liquid outflow path 114 is connected to the pipe 56 connected to the measuring unit 44.

If necessary, unevenness can be formed on the bottom surface of the groove 117. As the shape of the unevenness, for example, an asymmetric V-shaped groove called a chaotic mixer can be formed. By forming such unevenness, agitation occurs in the microchannel which tends to generate a laminar flow, and the efficiency of taking in target molecules via the porous membrane 121 described later is improved.

The porous membrane 121 covers the groove 117. The lid 112 is disposed on the porous membrane 121. The lid 112 is in close contact with the porous membrane 121 via a sealing member (for example, a rubber member). A groove 118 is formed on the surface of the lid 112 facing the porous membrane 121 in the same pattern as the groove 117.

The lid 112 is formed with an intake passage 115 connected to one end of the groove 118 and an exhaust passage 116 connected to the other end of the groove 118. The intake passage 115 is connected to the pipe 51 for taking in the sample atmosphere, and the exhaust passage 116 is connected to the pipe 52 for exhaust. The pipe 52 is provided with the intake and exhaust device 43.

By opening the valve 71 and the valve 72 toward the pipe 55 and the pipe 56, respectively, and driving the pump 12 and the pump 13, the organic solvent 100 stored in the organic solvent tank 41 is supplied from the liquid inflow path 113 to the groove 117. The organic solvent 100 does not permeate the porous membrane 121. Therefore, the organic solvent 100 does not flow into the groove 118 above the porous membrane 121. In the embodiment, only one of the pump 12 and the pump 13 may be used.

By driving the intake and exhaust device 43, the sample atmosphere taken into the pipe 51 from the atmosphere collection port 51a flows into the groove 118 from the intake passage 115. Further, the target molecules in the sample atmosphere permeate the porous membrane 121, enter the groove 117 to which the organic solvent is supplied, and are dissolved in the organic solvent flowing in the groove 117.

The organic solvent containing the target molecule in the groove 117 flows through the liquid outflow path 114 as it is and is supplied to the measuring unit 44.

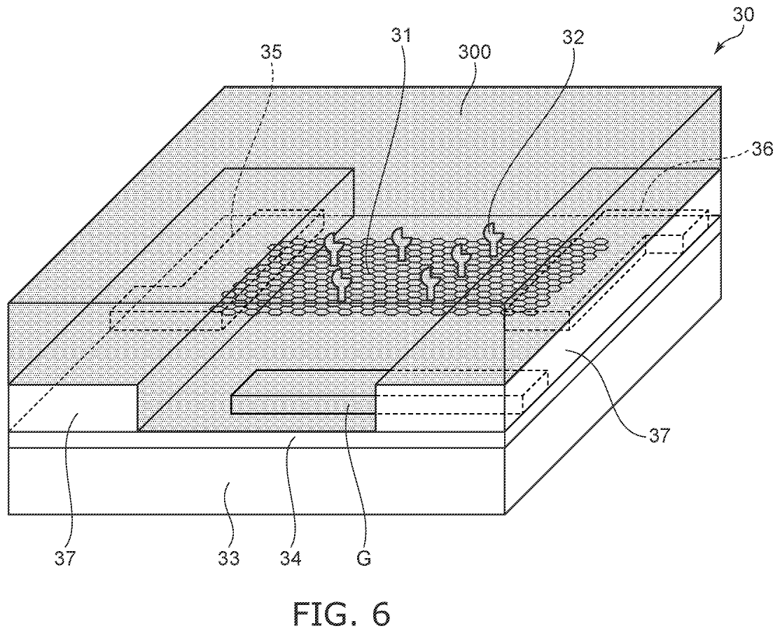

Next, an example of the sensor element 30 will be described with reference to FIG. 6.

The sensor element 30 is, for example, a charge detection element including a graphene film 31. The surface of the sensor element 30 (for example, the surface of the graphene film 31) is exposed to a sample solution 300 obtained by mixing an organic solvent containing the target molecules with an aqueous solution in the mixing unit 20.

The sensor element 30 has, for example, a field-effect transistor (FET) structure.

The sensor element 30 includes a substrate 33 and a base film 34 provided on the substrate 33. The graphene film 31 is provided on the base film 34. Alternatively, the graphene film 31 may be provided on the surface of the substrate 33 without providing the base film 34. Further, a circuit or a transistor (not shown) may be formed on the substrate 33.

As a material of the substrate 33, for example, silicon, silicon oxide, glass, or a polymer material can be used. The base film 34 is an insulating film such as a silicon oxide film. Further, the base film 34 can also have a function of a chemical catalyst for forming the graphene film 31.

Further, the sensor element 30 includes at least two electrodes (a first electrode 35 and a second electrode 36). One of the first electrode 35 and the second electrode 36 functions as a drain electrode, and the other functions as a source electrode.

The first electrode 35 and the second electrode 36 are covered with a protective insulating film 37. The protective insulating film 37 is, for example, aluminum oxide, silicon oxide, a polymer, or the like.

A gate wiring G is further formed on the base film 34 of the sensor element 30 and a part of the gate wiring G is exposed without being covered with the protective insulating film 37. The portion of the gate wiring G exposed from the protective insulating film 37 is made of gold, platinum, silver, silver/silver chloride laminated film, or the like.

Since the gate wiring G only needs to be in contact with the sample solution 300 in the vicinity of the sensor element 30, the gate wiring G does not necessarily have to be formed on the sensor element 30. For example, the gate wiring G may be formed on an element different from the sensor element 30 and exposed into the pipe through the window of the pipe as in the sensor element 30 to be brought into contact with the sample solution 300 or may be formed directly inside the pipe.

The graphene film 31 is provided between the first electrode 35 and the second electrode 36. The first electrode 35 and the second electrode 36 are in electrical contact with the graphene film 31. A current can flow between the first electrode 35 and the second electrode 36 through the graphene film 31.

The sensor element 30 further includes probe molecules 32 that selectively associate with the target molecules on the surface thereof. The probe molecule 32 is bonded or adsorbed on the surface of the graphene film 31.

The surface of the sensor element including the graphene film 31 is exposed in the flow path to which the sample solution 300 is supplied. The surface of the graphene film 31 and the probe molecule 32 are exposed to the sample solution 300.

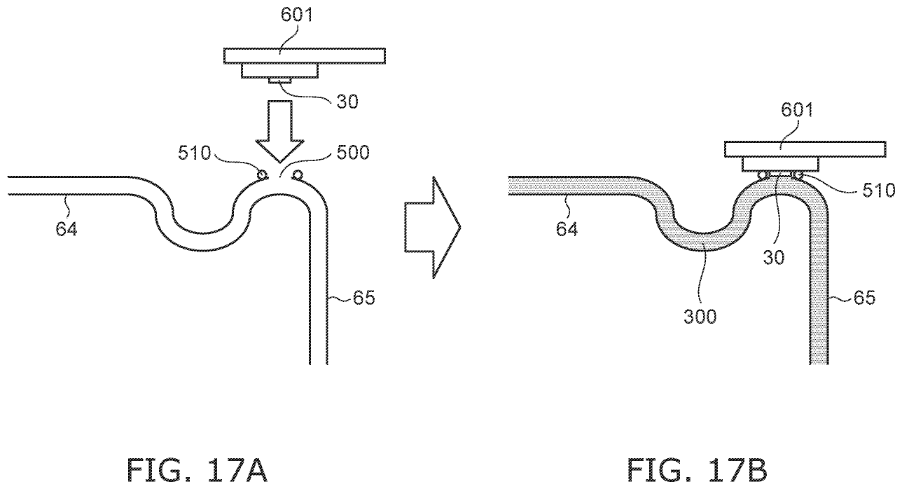

As shown in FIG. 17A, the pipe 64 and the pipe 65 have a window 500 opened at a sensor element mounting portion, and a packing 510 is formed on the outer periphery of the window 500. The sensor element 30 is mounted on a cartridge substrate 601. As shown in FIG. 17B, when the sensor element surface is installed so as to face the window 500 portion, the sensor element 30 is made airtight by the packing 510, and thus, the sensor element surface is exposed in the pipes 64 and 65. With such a form, the sensor element 30 can be attached and detached as a replacement part or a consumable part.

The sensor element 30 electrically detects that the probe molecule 32 has associated with the target molecule. When the probe molecule 32 recognizes and captures the target molecule, the target molecule is close to the surface of the graphene film 31, and thus, the electronic state of the graphene film 31 changes depending on, for example, the charge and polarization of the target molecule, and the electron attraction and donating property. By electrically detecting this, the presence and concentration of the target molecule can be found.

When the electronic state of the graphene film 31 is electrically detected, a desired gate potential is applied to the sample solution via the gate electrode, which enables the electrical characteristics of the graphene to be adjusted to a state of high sensitivity.

Alternatively, by measuring the current between the source and drain of graphene while scanning the gate potential, it is possible to measure the charge neutral point at which the carriers flowing in the graphene switch between holes and electrons and it is possible to know the state of charge injection into graphene.

If necessary, the surface of the graphene film 31 may be coated with an insulator. As the insulator, for example, a peptide β sheet, a phospholipid membrane, or the like can be used.

Next, the detection mechanism of the target molecule in the chemical sensor module of the embodiment will be described with reference to FIGS. 1, 7 to 10.

In the method for detecting target molecules using the chemical sensor module of FIG. 1, the first to ninth steps described below are performed in order.

(First Step)

The intake and exhaust device 43 is driven and the sample atmosphere is taken into the target molecule uptake unit 10 through the pipe 51. Further, the valve 71 is switched to a state in which the pipe 54 and the pipe 55 communicate with each other and the organic solvent is supplied from the organic solvent tank 41 to the target molecule uptake unit 10. In the target molecule uptake unit 10, the target molecule in the sample atmosphere is dissolved in the organic solvent.

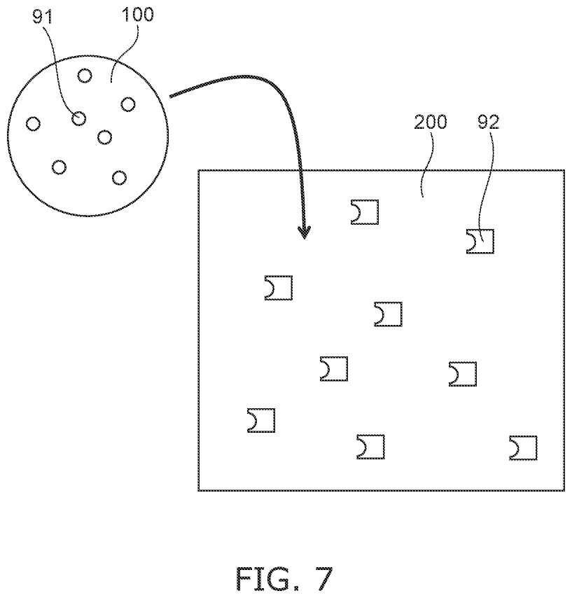

FIG. 7 schematically shows a target molecule 91 dissolved in the organic solvent 100. The hydrophobic target molecule 91 is sparingly soluble in an aqueous solution, but is soluble in the organic solvent 100 and dispersed in the organic solvent 100. Therefore, the hydrophobic target molecule 91 can be efficiently incorporated into the liquid from the air. For example, the target molecule 91 is limonene and the organic solvent 100 is ethanol or DMSO.

(Second Step)

The valve 72 is switched to a state in which the pipe 56 and the pipe 57 communicate with each other and the organic solvent in which target molecules are dissolved is supplied from the target molecule uptake unit 10 to the measuring unit 44. Further, the valve 76 is opened to supply the aqueous solution from the aqueous solution tank 42 to the measuring unit 45.

As shown in FIG. 7, an aqueous solution 200 contains labeled molecules 92 that have an affinity for the target molecule 91. The labeled molecule 92 is hydrophilic and is dissolved and dispersed in the aqueous solution 200. The aqueous solution 200 is, for example, a phosphate buffer, a HEPES buffer, a Tris-hydrochloride buffer, or the like. The number of the labeled molecules 92 is larger than the number of the target molecules 91. The labeled molecule 92 is any one among a molecule having a molecular weight larger than that of the target molecule 91, a charged molecule, and a polarized polar molecule. The labeled molecule 92 is, for example, arginine, arginine methyl ester, arginine amide, nucleic acid aptamer, or peptide.

(Third Step)

The valve 75 is opened to supply the mixing unit 20 with a first predetermined amount of the organic solvent (containing the target molecules) measured in the measuring unit 44. Further, the valve 78 is opened to supply the mixing unit 20 with a second predetermined amount of the aqueous solution measured by the measuring unit 45. As a result, in the mixing unit 20, a sample solution is prepared in which the first predetermined amount of the organic solvent containing the target molecules is mixed with the second predetermined amount of the aqueous solution.

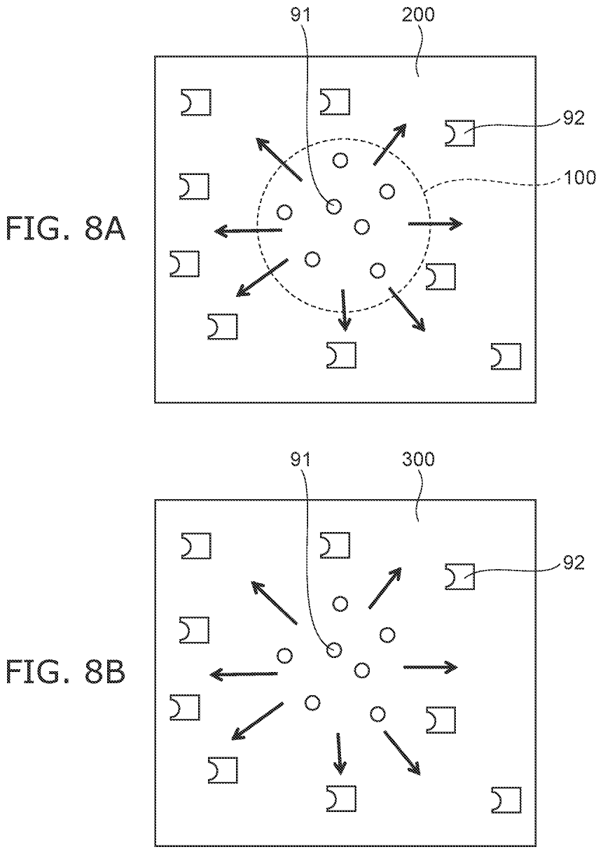

As shown in FIG. 8A, the hydrophilic organic solvent 100 diffuses in the aqueous solution 200 and the organic solvent 100 and the aqueous solution 200 are admixed. Then, as shown in FIG. 8B, the target molecule 91 rapidly dissipates into the aqueous solution 200 and the sample solution 300 is obtained. Since the target molecule 91 is hydrophobic, the target molecule 91 is left in an unstable state in the sample solution 300. Although the target molecule 91 is hydrophobic, the target molecule 91 is mixed with the aqueous solution 200 in a state of being dispersed in the hydrophilic organic solvent 100. Since the hydrophilic organic solvent 100 is mixed with the aqueous solution 200, the target molecule 91 can be efficiently dispersed into the aqueous solution 200 with less energy than when the hydrophobic target molecule 91 is directly incorporated into the aqueous solution 200 without the intervention of the organic solvent 100.

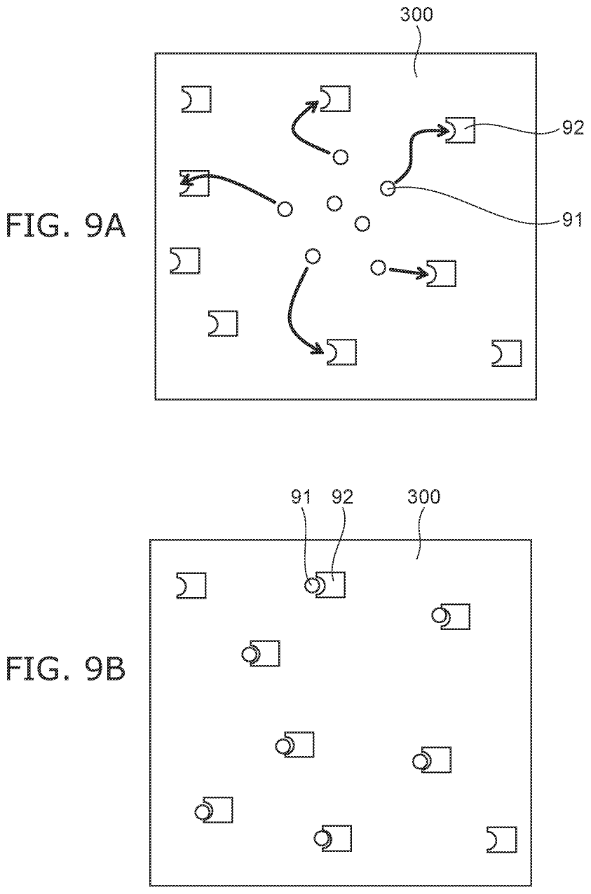

The labeled molecules 92 are dispersed in the aqueous solution 200. Therefore, as shown in FIG. 9A, the labeled molecules 92 are also dispersed in the sample solution 300. The sample solution 300 is an aqueous solution obtained by diluting the organic solvent 100 with the aqueous solution 200 and the hydrophobic target molecules 91 in the sample solution 300 are in an unstable state. The unstable target molecule 91 associates with the nearby labeled molecule 92, as shown in FIG. 9B. For example, limonene as the target molecule 91 and arginine amide as the labeled molecule 92 are bonded by π-π interaction to form an aggregate.

(Fourth Step)

The sample solution is supplied from the mixing unit 20 to the sensor element 30 through the pipe 64 by opening the valve 77. Then, in the sensor element 30, a signal (for example, an electric signal) corresponding to the target molecule in the sample solution is measured.

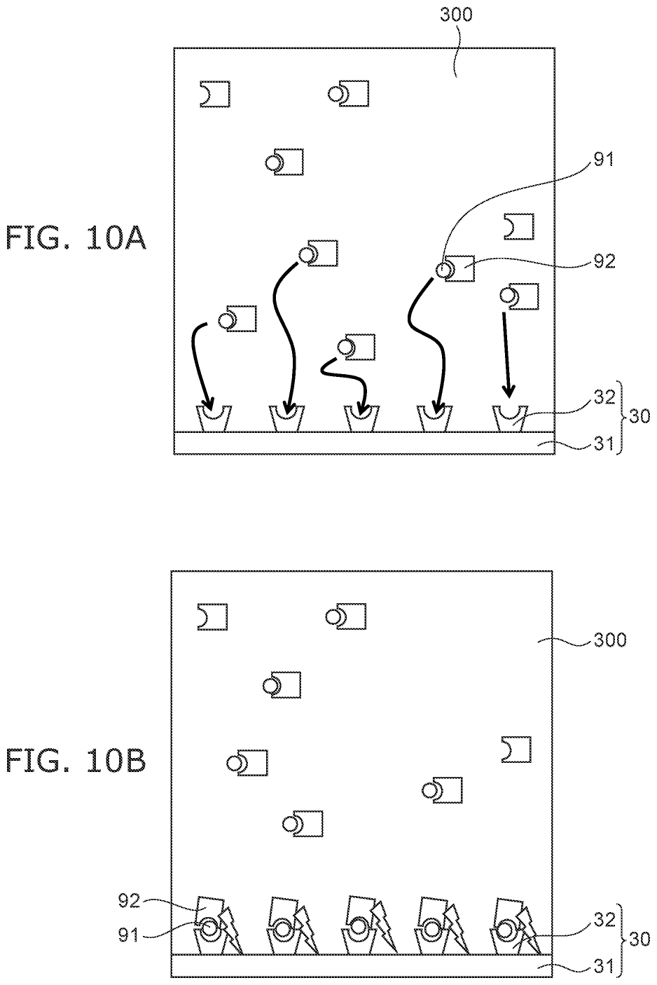

As shown in FIG. 10A, the target molecule 91 is captured by a probe molecule 32 of the sensor element 30 and becomes close to the surface of the graphene film 31. The presence or concentration of the target molecule 91 in the sample solution 300 can be detected by detecting the change in the electronic state of the graphene film 31 due to the proximity of the target molecule 91 to the graphene film 31 (for example, the proximity of the charge of the target molecule 91).

When the target molecule 91 is uncharged and has a small molecular weight (for example, when the molecular weight is 300 or less), it may be difficult to detect a change in the electronic state of the graphene film 31 caused by the proximity to the graphene film 31. According to the embodiment, since the target molecule 91 is associated with the labeled molecule 92, the labeled molecule 92 is close to the graphene film 31 as shown in FIG. 10B when the target molecule 91 is captured by the probe molecule 32. Here, when the labeled molecule 92 has a strong charge such as arginine amide, or when the labeled molecule 92 has a large molecular weight (for example, 500 or more) such as a nucleic acid or a peptide, the sensor element 30 detects a change in the electronic state of the graphene film 31 caused by the proximity of the labeled molecule 92 (for example, a change in the distribution of ions at the solution interface due to the proximity of the charge of the labeled molecule 92 and the proximity of the large labeled molecule 92). Thereby, even if the detection is difficult with the proximity of only the target molecule 91, the presence or concentration of the target molecule 91 in the sample solution 300 can be detected by detecting the proximity of the labeled molecule 92.

The detection target is the target molecule 91 that was present in the sample atmosphere and the probe molecule 32 that can capture the target molecule 91 is selected. The labeled molecule 92 is selected so as not to interfere with the capture of the target molecule 91 by the probe molecule 32 and to associate with the target molecule 91 so as not to cover the site where the target molecule 91 binds to the probe molecule 32.

(Fifth Step)

The valve 73 is opened and the rest of the organic solvent to which the sample atmosphere in the target molecule uptake unit 10 is exposed is discharged from the target molecule uptake unit 10 through the pipe 53. Further, the valve 79 is opened and the rest of the sample solution in the mixing unit 20 is discharged from the mixing unit 20 through the pipe 66.

(Sixth Step)

The valve 71 and the valve 72 are opened toward the pipe 55 and the pipe 56, respectively, and the valve 74 is further opened to drain the organic solvent from the organic solvent tank 41 to the pipe 59 via the target molecule uptake unit 10 and the measuring unit 44. The target molecule uptake unit 10 and the measuring unit 44 are washed with the organic solvent in the organic solvent tank 41 in which the sample atmosphere is not exposed. The target molecule is discharged from the target molecule uptake unit 10 and the measuring unit 44.

(Seventh Step)

The valve 71 and the valve 72 are opened toward the pipe 58 and the organic solvent is supplied from the organic solvent tank 41 to the measuring unit 44 through the pipe 58. The organic solvent is supplied from the organic solvent tank 41 to the measuring unit 44 without passing through the target molecule uptake unit 10. Further, the valve 76 is opened to supply the aqueous solution from the aqueous solution tank 42 to the measuring unit 45 through the pipe 62.

(Eighth Step)

The valve 75 is opened to supply the mixing unit 20 with the same first predetermined amount of the organic solvent (which does not contain the target molecule) as measured in the measuring unit 44. Further, the valve 78 is opened to supply the mixing unit 20 with the same second predetermined amount of the aqueous solution as measured by the measuring unit 45. As a result, in the mixing unit 20, a control solution is prepared in which the first predetermined amount of the organic solvent containing no target molecule is mixed with the second predetermined amount of the aqueous solution.

(Ninth Step)

The control solution is supplied from the mixing unit 20 to the sensor element 30 through the pipe 64 by opening the valve 77. By comparing a measured signal from the sensor element 30 exposed to the control solution containing no target molecule with a measured signal from the sensor element 30 exposed to the sample solution, it is possible to detect the target molecule with high accuracy corrected for disturbance noise if the sample solution contains the target molecule.

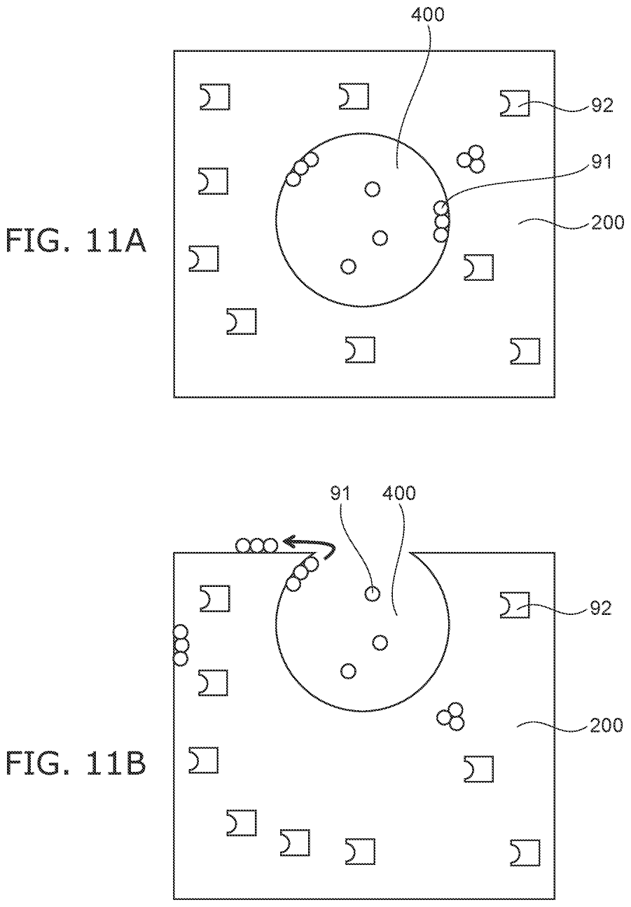

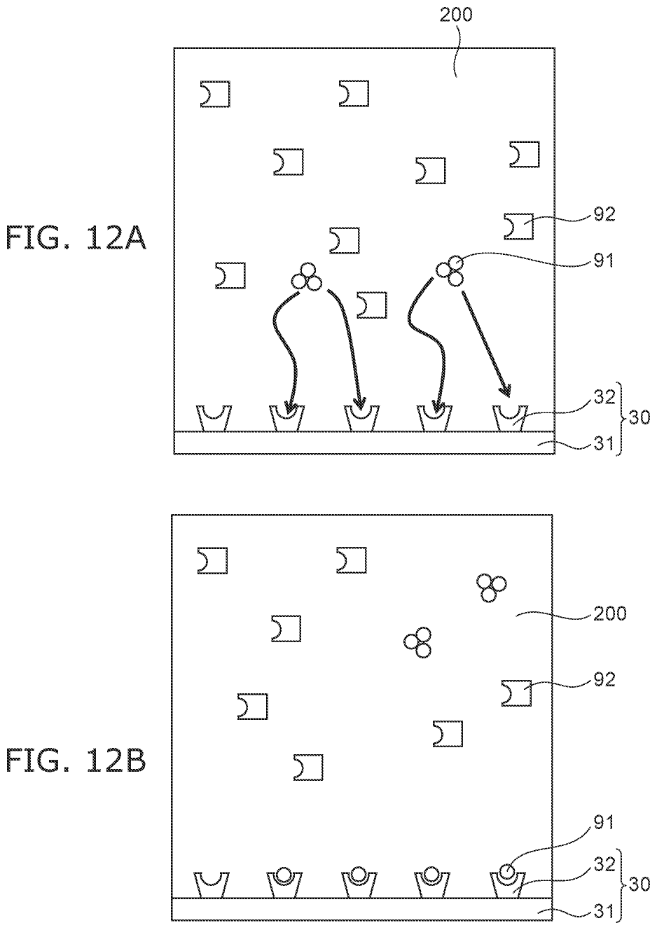

FIGS. 11A to 12B are schematic views showing the detection mechanism of the target molecule in a comparative example.

In this comparative example, as shown in FIG. 11A, the hydrophobic target molecule 91 is incorporated into the aqueous solution 200 without the intervention of an organic solvent. For example, the sample atmosphere containing the target molecules 91 is incorporated into the aqueous solution 200 as bubbles 400. The hydrophobic target molecule 91 is likely to segregate on the surface of the bubble 400. The labeled molecule 92 dispersed in the aqueous solution 200 does not have a binding force to the target molecule 91 enough to draw the target molecule 91 in the bubble 400 into the liquid. Further, when the hydrophobic target molecules 91 are incorporated into the solution, the target molecules 91 tend to aggregate.

As shown in FIG. 11B, when the bubble 400 bursts, the target molecules 91 segregated on the surface of the bubble 400 move to the liquid surface of the aqueous solution 200. When the bubbles 400 touch the wall surface of a container storing the aqueous solution 200, the target molecules 91 move to the wall surface. In addition, the target molecules 91 incorporated into the liquid maintain a thermodynamically more stable aggregated state than the dispersed state.

FIG. 12A shows a state in which the aqueous solution 200 incorporating the target molecule 91 is exposed to the surface of the sensor element 30. Then, as shown in FIG. 12B, the target molecule 91 slightly incorporated into the liquid is captured by the probe molecule 32. However, when the target molecule 91 is uncharged and has a small molecular weight like limonene, it is difficult to detect a change in the electronic state of the graphene film 31 caused by the proximity to the graphene film 31.

According to the embodiment, the hydrophobic target molecule is concentrated in an organic solvent and incorporated and the organic solvent aqueous solution (sample solution) incorporating the target molecule is exposed to the surface of the sensor element. Therefore, the detection sensitivity of the target molecule can be increased as compared with the case where the hydrophobic target molecule is directly incorporated into the aqueous solution.

Furthermore, if a labeled molecule having an affinity for the target molecule is dissolved in the aqueous solution, the target molecule is bonded to the labeled molecule and labeled when the organic solvent containing the target molecule is diluted with the aqueous solution. Thus, the detection signal of the target molecule is amplified.

In the case of a hydrophobic target molecule, hydrophobic interaction is a major factor as a force to bind to the probe molecule. Hydrophobic interaction is a force that makes hydrophobic groups approach each other and is weakened in an organic solvent having a high affinity for hydrophobic groups. Therefore, when the organic solvent incorporating the target molecule is exposed to the surface of the sensor element without dilution with an aqueous solution, the ability of the probe molecule to capture the target molecule is reduced.

In addition, molecules derived from living bodies (for example, peptides, DNA aptamers, and the like) function in an aqueous solution. Therefore, when a molecule derived from a living body is used as a probe molecule and an organic solvent is exposed to the surface of the sensor element, the structure of the probe molecule derived from the living body is changed or destroyed, and the ability of the probe molecule to capture the target molecule is reduced.

Also, since graphene is hydrophobic, graphene has a high affinity with organic solvents, and if the organic solvent is exposed to the surface of the sensor element, graphene may be damaged. Due to the damage, there is a concern that the organic solvent may infiltrate the adhesive surface between the graphene film and the underlying insulating film and the interface between the source and drain electrodes and the protective insulating film that covers the source and drain electrodes, causing the graphene film and the protective insulating film to peel off.

In the embodiment, the sample solution, which is an aqueous solution obtained by diluting an organic solvent with an aqueous solution, is exposed to the surface of the sensor element, and thus, the above problem does not occur.

In the configuration of the chemical sensor module shown in FIG. 1, the incorporation of the target molecule from the gas phase (in the air) into the organic solvent may be performed while the control solution is measured. After the first step described above, the seventh to ninth steps are performed. Then, after the ninth step, the second to sixth steps are performed.

That is, after the sample atmosphere is exposed to the organic solvent in the target molecule uptake unit 10, the organic solvent is supplied from the organic solvent tank 41 to the mixing unit 20 without passing through the target molecule uptake unit 10, and the aqueous solution is supplied to the mixing unit 20. Then, the control solution prepared by the mixing unit 20 is supplied to the sensor element 30 to measure the control solution. After that, the organic solvent is supplied from the target molecule uptake unit 10 to the mixing unit 20 and the aqueous solution is supplied to the mixing unit 20. Then, the sample solution prepared by the mixing unit 20 is supplied to the sensor element 30 to measure the sample solution.

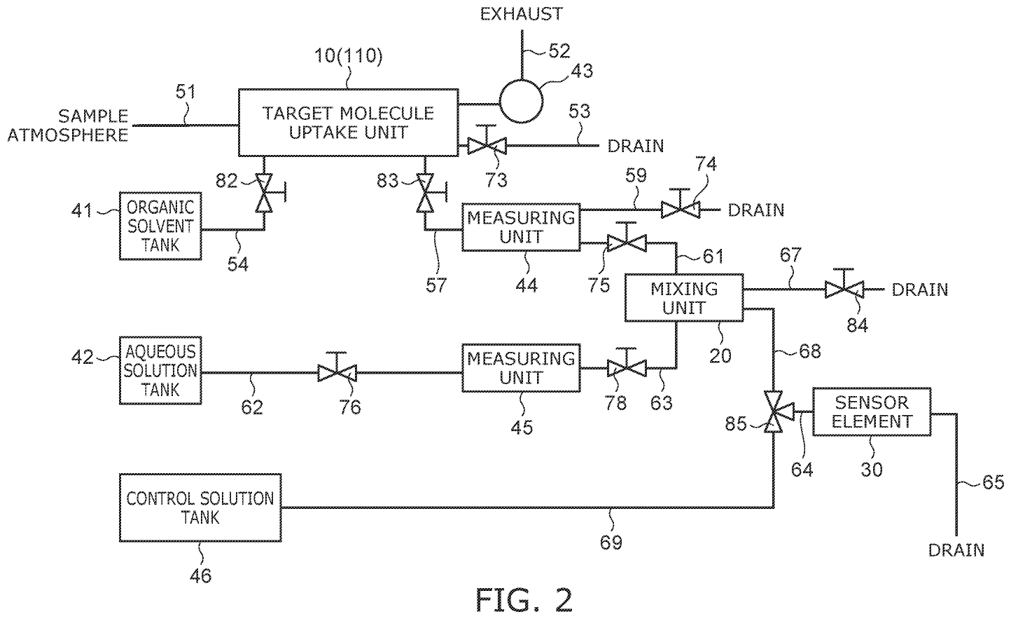

FIG. 2 is a schematic configuration diagram showing another example of the chemical sensor module of the embodiment.

The chemical sensor module of FIG. 2 differs from the chemical sensor module of FIG. 1 in the following points.

The organic solvent tank 41 is connected to the target molecule uptake unit 10 via the pipe 54 and a valve 82 is connected to the pipe 54. The target molecule uptake unit 10 is connected to the measuring unit 44 via the pipe 57 and a valve 83 is connected to the pipe 57. There is no system for directly supplying the organic solvent from the organic solvent tank 41 to the measuring unit 44 without passing through the target molecule uptake unit 10.

The mixing unit 20 is connected to the sensor element 30 via a pipe 68, a valve 85, and the pipe 64.

A control solution tank 46 is provided as a supply source of the control solution. The control solution tank 46 is connected to the sensor element 30 via a pipe 69, the valve 85, and the pipe 64.

The valve 85 is a three-way valve and can switch between a first state of communicating between the mixing unit 20 and the sensor element 30 and blocking between the control solution tank 46 and the sensor element 30, and a second state of blocking between the mixing unit 20 and the sensor element 30 and communicating between the control solution tank 46 and the sensor element 30.

In the method for detecting a target molecule using the chemical sensor module of FIG. 2, the first to fourth steps described below are performed in order.

(First Step)

The intake and exhaust device 43 is driven and the sample atmosphere is taken into the target molecule uptake unit 10 through the pipe 51. Further, the valve 82 is opened to supply the organic solvent from the organic solvent tank 41 to the target molecule uptake unit 10. As a result, in the target molecule uptake unit 10, the target molecule in the sample atmosphere is dissolved in the organic solvent.

The valve 74 is opened and the rest of the organic solvent in the measuring unit 44 is discharged from the measuring unit 44 through the pipe 59. The valve 84 is opened and the rest of the sample solution in the mixing unit 20 is discharged from the mixing unit 20 through a pipe 67.

The valve 85 is switched to the second state, the control solution is supplied from the control solution tank 46 to the sensor element 30, and the control solution is measured.

(Second Step)

In step 2, the measurement of the control solution by the sensor element 30 is continued. Further, in step 2, the valve 83 is opened and the organic solvent to which the sample atmosphere is exposed is supplied from the target molecule uptake unit 10 to the measuring unit 44 through the pipe 57. Further, the valve 76 is opened to supply the aqueous solution from the aqueous solution tank 42 to the measuring unit 45.

(Third Step)

In step 3, the measurement of the control solution by the sensor element 30 is continued. Further, in step 3, the valve 75 is opened to supply the mixing unit 20 with a first predetermined amount of the organic solvent measured by the measuring unit 44. Further, the valve 78 is opened to supply the mixing unit 20 with a second predetermined amount of the aqueous solution measured by the measuring unit 45. As a result, in the mixing unit 20, a sample solution is prepared in which the first predetermined amount of the organic solvent is mixed with the second predetermined amount of the aqueous solution.

(Fourth Step)

The valve 85 is switched to the first state, the supply of the control solution to the sensor element 30 is stopped and the sample solution is supplied from the mixing unit 20 to the sensor element 30. Then, in the sensor element 30, a signal corresponding to the target molecule in the sample solution is measured.

According to the configuration of FIG. 2, the sample solution can be prepared in the mixing unit 20 during the measurement of the control solution by the sensor element 30. Then, by switching the valve 85, it is possible to quickly switch to the measurement of the sample solution.

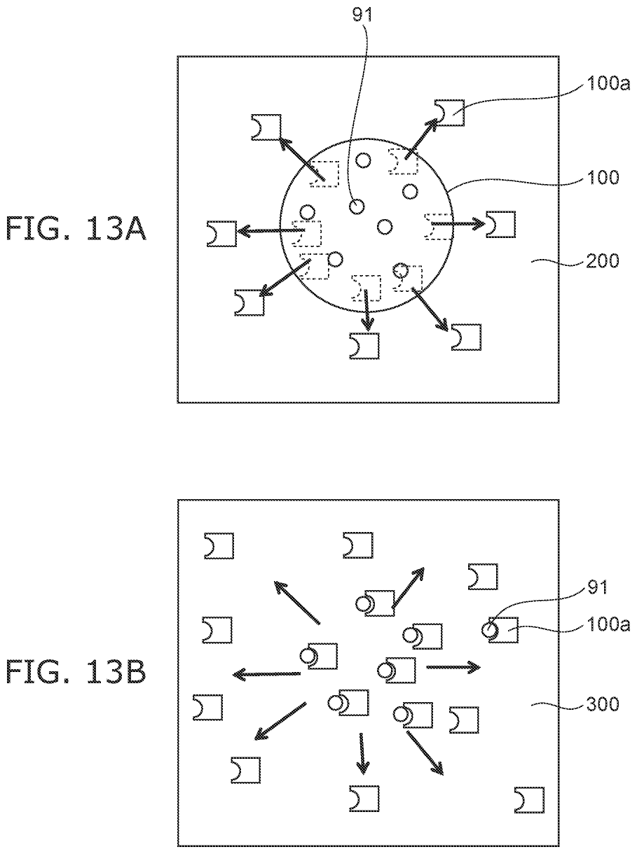

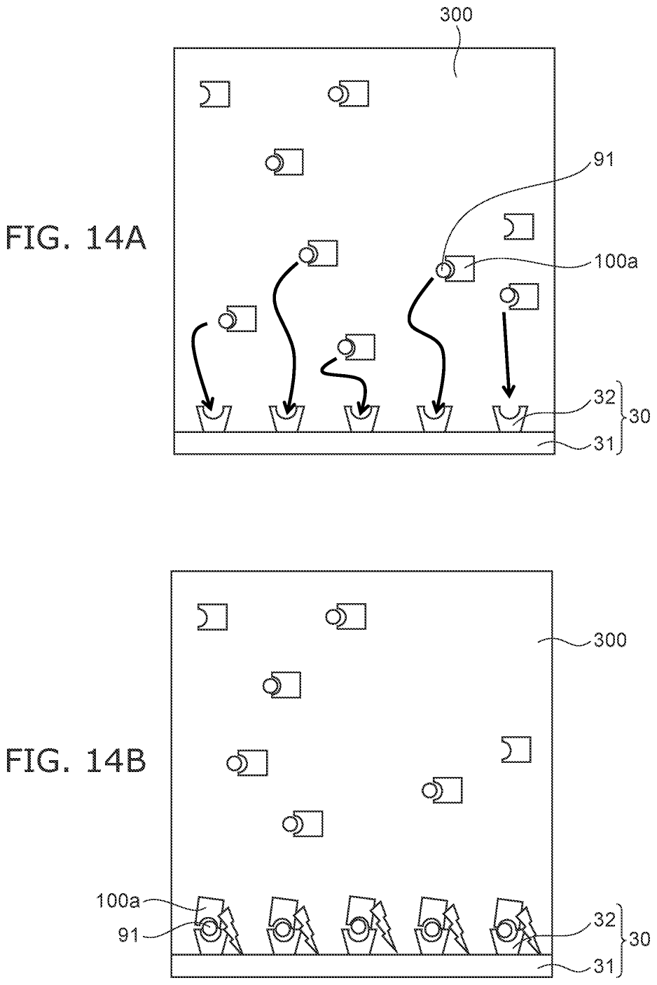

Next, with reference to FIGS. 13A to 14B, another example of the detection mechanism of the target molecule using the chemical sensor module of the embodiment will be described.

In the target molecule uptake unit 10 described above, the target molecule in the sample atmosphere is dissolved in an organic solvent and dispersed at the molecular level. For example, the target molecule is limonene and the organic solvent is DMSO.

In the mixing unit 20, the organic solvent containing the target molecule and the aqueous solution are mixed to prepare a sample solution. For example, the aqueous solution is a HEPES (4-(2-hydroxyethyl)-1-piperazineethanesulfonic acid)) buffer.

As shown in FIG. 13A, the hydrophilic organic solvent 100 diffuses in the aqueous solution 200, and the organic solvent 100 and the aqueous solution 200 are admixed. Then, as shown in FIG. 13B, the target molecule 91 bonded to an organic solvent molecule 100a is released in the aqueous solution 200 and the sample solution 300 is prepared.

The sample solution is supplied to the sensor element 30. Then, in the sensor element 30, a signal (for example, an electric signal) corresponding to the target molecule 91 in the sample solution is measured.

Since the hydrophobic target molecule 91 is unstable when the target molecule 91 is released alone in an aqueous solution, the target molecule 91 remains bonded to the organic solvent molecule 100a as shown in FIG. 14A. When the target molecule 91 is captured by the probe molecule 32 of the sensor element 30, the organic solvent molecule 100a bonded to the target molecule 91 is close to the graphene film 31 as shown in FIG. 14B. For example, when the organic solvent molecule 100a is DMSO, electrons are injected into the graphene film 31 from an electron-donating lone pair of DMSO in the vicinity of the graphene film 31. By detecting the change in the electronic state of the graphene film 31 at this time, the presence or concentration of the target molecule 91 in the sample solution 300 can be detected. In this case, the organic solvent molecule 100a itself functions as a labeled molecule having an affinity for the target molecule 91. Even if the target molecule 91 is a non-charged and non-polar small molecule such as limonene, the presence and concentration of the target molecule 91 in the sample solution 300 can be detected by detecting a labeled molecule (organic solvent molecule 100a) having a charge or polarity or larger than the target molecule 91.

Next, an experiment for detecting limonene (target molecule) according to the embodiment of the invention will be described.

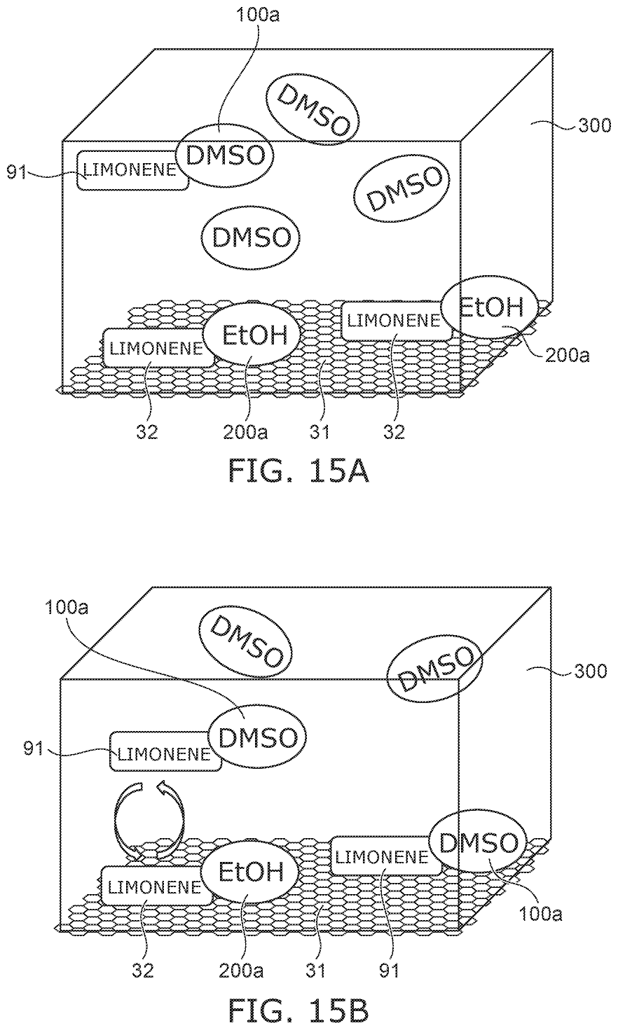

An ethanol solution in which limonene was dissolved was mixed with HEPES buffer and dropped onto the graphene film of the sensor element. As shown in FIG. 15A, the hydrophobic limonene is non-specifically adsorbed on the graphene film 31 together with an ethanol molecule 200a to become the probe molecule 32. That is, the probe molecule 32 having the same molecular structure as the target molecule 91 is provided on the surface of the graphene film 31 exposed to the sample solution in the sensor element.

Next, the above mixture of ethanol solution and HEPES buffer was replaced with an aqueous solution containing DMSO at a concentration of 2% in 1 mM HEPES buffer. That is, after limonene, which is the probe molecule 32, is fixed or adsorbed on the surface of the sensor element in a state where limonene does not coexist with the organic solvent, the surface of the sensor element is kept covered with the aqueous solution.

Next, after dissolving limonene as the target molecule 91 in DMSO as an organic solvent, the organic solvent solution was mixed with the above aqueous solution to prepare the sample solution 300. The concentration of DMSO was adjusted to 2%. In the sample solution 300, the target molecule (limonene) 91 is bonded to the organic solvent molecule (DMSO molecule) 100a.

When the DMSO aqueous solution containing the target molecule 91 is replaced with the DMSO aqueous solution not containing the target molecule 91, the limonene of the probe molecule 32 exchanges with the limonene of the target molecule 91, as shown in FIG. 15B. Alternatively, due to the affinity of limonene, the limonene of the target molecule 91 is adsorbed on the limonene of the probe molecule 32. The sensor element detects the proximity of the organic solvent molecule (DMSO molecule) 100a bonded to the target molecule (limonene) 91 adsorbed on the graphene film 31 in exchange for the probe molecule (limonene) 32. Alternatively, the proximity of the organic solvent molecule (DMSO molecule) 100a bonded to the target molecule (limonene) 91 adsorbed on the probe molecule (limonene) 32 is detected.

The aqueous solution in which the organic solvent in which the target molecule 91 is dissolved is mixed is a DMSO solution, and a constant amount (adsorption equilibrium amount) of DMSO is originally adsorbed on the surface of the graphene film 31 in this DMSO solution. Further, since the organic solvent molecule (DMSO molecule) 100a bonded to the target molecule 91 approaches the surface of the graphene film 31 by adsorbing the target molecule 91 to the graphene film 31, the amount of DMSO on the surface of the graphene film 31 increases. Since DMSO has an electron-donating lone pair, electrons are injected into the graphene film 31. When the drain current flowing through the graphene film 31 is due to hole conduction, the conduction carrier (hole) is reduced by the injection of electrons, so that the drain current is lowered.

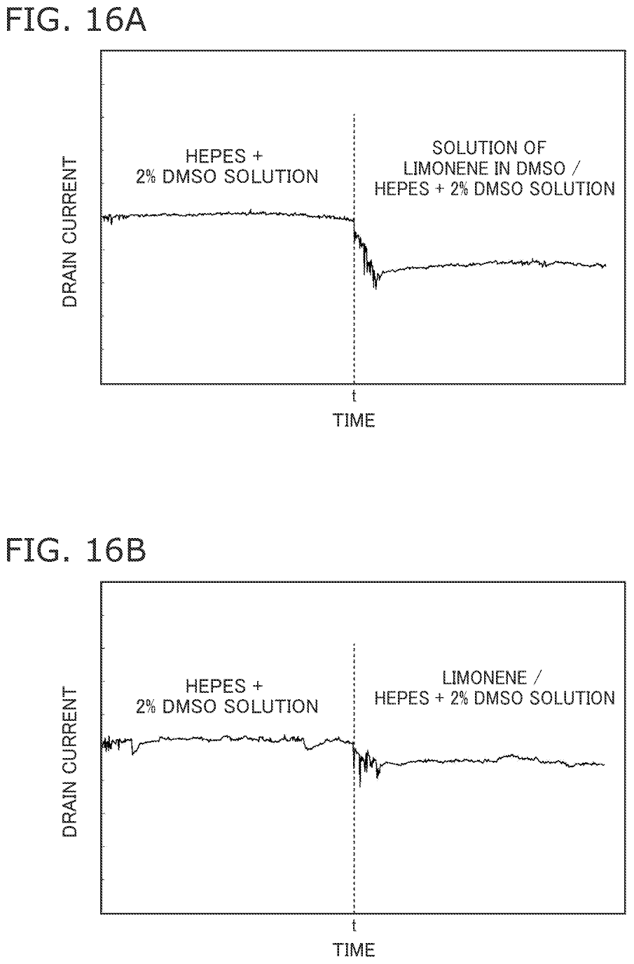

FIG. 16A is a graph showing a detection result of limonene by an experiment of the embodiment. FIG. 16B is a graph showing a detection result of limonene by a control experiment. In both FIGS. 16A and 16B, the horizontal axis represents time and the vertical axis represents drain current (more accurately, the ratio of the drain current value after aging to the initial drain current value).

In the limonene detection experiment according to the embodiment, as described above, after dissolving limonene in DMSO in advance, an aqueous solution mixed with 1 mM HEPES buffer (at this time, the DMSO concentration is adjusted to 2%) is used. At time t, it was replaced with 1 mM HEPES buffer without limonene and containing 2% DMSO.

In the control experiment, an aqueous solution obtained by mixing DMSO with an aqueous solution containing DMSO at a concentration of 2% in 1 mM HEPES buffer was replaced with a 1 mM HEPES buffer containing 2% DMSO without limonene at time t.

Comparing the experimental results according to the embodiment shown in FIG. 16A with the results of the control experiment shown in FIG. 16B, the experiment according to the embodiment shows that the amount of the change in the drain current after time t is large, and limonene can be detected with high sensitivity.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modification as would fall within the scope and spirit of the inventions.

What is claimed is:

1. A chemical sensor module comprising:
a target molecule uptake unit that exposes a sample atmosphere containing hydrophobic target molecules to a hydrophilic organic solvent;
a mixing unit that mixes the organic solvent containing the target molecules with an aqueous solution to prepare a sample solution; and
a sensor element having a surface exposed to the sample solution.

2. The module according to claim 1, wherein
either the aqueous solution or the organic solvent contains a labeled molecule having an affinity for the target molecule, and
the sensor element detects a proximity of the labeled molecule.

3. The module according to claim 2, wherein
the labeled molecule is any one among a molecule having a molecular weight larger than a molecular weight of the target molecule, a charged molecule, and a polarized polar molecule.

4. The module according to claim 1, wherein
the organic solvent is selected from a group consisting of lower alcohols, DMSO (dimethyl sulfoxide), DMF (N,N-dimethylformamide), acetone, and acetonitrile.

5. The module according to claim 1, wherein
the aqueous solution is a phosphate buffer or a HEPES (4-(2-hydroxyethyl)-1-piperazineethanesulfonic acid)) buffer.

6. The module according to claim 1, wherein
the sensor element is a charge detection element containing graphene.

7. The module according to claim 1, wherein
the sensor element further includes probe molecules that associate with the target molecules on the surface.

8. The module according to claim 1, wherein
the sensor element further includes probe molecules having a same molecular structure as the target molecules on the surface.

9. The module according to claim 8, wherein
the probe molecule fixed or adsorbed on the sensor element is provided on the surface of the sensor element exposed to the sample solution.

10. A method for detecting hydrophobic target molecules, comprising:
exposing a sample atmosphere containing hydrophobic target molecules to a hydrophilic organic solvent to dissolve the target molecules in the organic solvent;
mixing the organic solvent containing the target molecules with an aqueous solution to prepare a sample solution; and
exposing the sample solution to the surface of a sensor element.

11. The method according to claim 10, wherein
either the aqueous solution or the organic solvent contains labeled molecules having an affinity for the target molecules.
